(19) United States
(12) Patent Application Publication
(10) Pub. No.: US 2020/0354868 A1
(43) Pub. Date: Nov. 12, 2020

(54) Containment closure method for containing friable material and contained friable material

(71) Applicant: Preco, Inc., Somerset, WI (US)

(72) Inventors: Ken Arvey, Emerald, WI (US); Heather Landin, Baldwin, WI (US); Terry Rusch, Vadnais Heights, MN (US); Daniel B. Miller, Roberts, WI (US); Kimberly Hill, South St. Paul, MN (US)

(21) Appl. No.: 16/868,988

(22) Filed: May 7, 2020

Related U.S. Application Data
(60) Provisional application No. 62/844,474, filed on May 7, 2019.

(51) Int. Cl. D04H 1/4218; B32B 5/26; B32B 5/06; D04H 1/02; B32B 5/02; D04H 1/12; D04H 1/425; D04H 1/4266; A61L 27/10; A61L 27/20; A61L 15/18; A61L 15/28

(52) U.S. Cl. CPC D04H 1/4218; D10B 2201/02; B32B 5/06; D04H 1/02; B32B 5/022; D04H 1/12; D04H 1/425; D04H 1/4266; A61L 27/10; A61L 27/20; A61L 15/18; A61L 15/28; B32B 2262/101; B32B 2262/062; B32B 2535/00; D10B 2509/022; D10B 2101/06; B32B 5/26

(57) ABSTRACT

A method for forming an article where a friable material is supported by or enclosed by a non-friable material. The article comprises one or more layers of non-friable fibers surrounding the friable material fibers wherein at least some of the non-friable fibers are oriented in a Z plane and at least some of the non-friable fibers are oriented in an XY plane. The fibers are oriented in the Z plane via a felting process such as needle punching through the assembly of the article. The resulting article is a non-woven article even where one or more layer for the article was provided as woven or non-woven layer of fibers.

### CROSS-REFERENCE TO RELATED APPLICATION

The present application claims priority to and the benefit of U.S. provisional application Ser. No. 62/844,474, filed on May 7, 2019, the contents of which are hereby incorporated in their entirety.

### BACKGROUND

The present disclosure relates to bioactive material assemblies and more specifically to articles configured for use in bandages for wounds, packing for primary wound treatment in the field, permanent wound treatments, packing, in vivo scaffolding or combinations thereof where the assembly comprises non-friable materials supporting or encapsulating a friable material.

Regenerative medicine is a field of medicine related to regenerating damaged tissues in the body rather than replacing the tissues such as by graft or transplant. Procedures for in vivo regeneration of tissue generally utilize a scaffold, or support, for allowing and/or encouraging the regeneration of the diseased, damaged or missing tissue in the selected area. More recently, these scaffolds are a kind of template for the regeneration such that these scaffolds have preselected dimensions and may be impregnated with cells or other growth factors for encouraging tissue regeneration.

Scaffolds for tissue and bone regeneration must be highly porous so as to support cellular proliferation and allow for diffusion of nutrients as well as waste products. Prior art methods of forming porous scaffolds with materials such as Bioglass, include use of a porogenic agent, cellulose nanocrystals as a template and polymer swelling followed by a freeze-drying process. These methods produce a biodegradable scaffold having thick pore walls and low free volume for cell diffusion. As such, such scaffolds take longer to biodegrade.

Bioglass, is a glass composed within a range of 45 wt % SiO₂, 24.5 wt % CaO, 24.5 wt % Na₂O, and 6.0 wt % P₂O₅. Glasses are non-crystalline amorphous solids that are commonly composed of silica-based materials with other minor additives. Bioglass has a high ratio of calcium to phosphorus (5:1 molar ratio) which promotes the formation of apatite crystals and the composition of Bioglass is optimal in biomedical applications as Bioglass is compositionally similar to hydroxyapatite (HA) which is the mineral component of bone. As such, Bioglass is used in scaffolding for its ability to be integrated with living bone. While Bioglass is able to chemically bond with or stimulate the growth of tissue on itself and is used therapeutically with bone and is biodegradable, it is known to be brittle. Further, it is known that Bioglass is difficult to form into porous three-dimensional scaffolds as the material is difficult to sinter into a dense network.

### SUMMARY

An aspect of the present disclosure relates to a method for producing an article with a friable material enclosed therein. The method includes providing a first layer comprising non-friable fibers; providing a second layer comprising friable material on top of the first layer; and providing a third layer comprising non-friable fibers on top of the first and second layer to form an assembly. The article is then needle punched by pushing a felting needle repeatedly and entirely through at least the first and third layer to the first and third layer together with the second layer therebetween such that the first and third layer are secured around the second layer.

The first and third layer may each have a width and a length greater than a width and a length of the second layer such that the first and third layers are secured together to form a perimeter around the friable material to surround or enclose the friable material.

The biocompatible fibers of the first and third layer are the same or different biocompatible fibers.

A portion of the surface area of the assembly or substantially the surface area of the assembly may be felted to form an article. Felting may be done by needle punching.

Yet another aspect of the present disclosure relates to a method of producing a non-woven cohesive fiber article by providing one or more layers of non-friable fibers and a layer of friable fibers, within, wherein the non-friable fibers are substantially oriented in the XY plane and pushing a felting needle through a thickness defined by the layer or layers of the non-friable fibers and the layer of the friable fibers to push at least a portion of the non-friable fibers to change the orientation of the portion of the non-friable to orientation in the Z plane.

Yet another aspect of the present disclosure relates to a method of producing a felted fiber article wherein friable and non-friable material are mixed together, for example in a drum mixer, and provided in one or more layers. The layer(s) is/are needle felted such that the non-friable fibers contain the friable material within the article.

In one or more methods described herein, pushing the felting needle comprises needle felting the all layers of non-friable fibers together by pushing non-friable fibers through all layers and into orientation along a Z-axis to enmesh the non-friable fibers of all the respective layers and securing all layers together.

In one or more methods described herein, the friable material comprises bioactive glass, unmodified chitosan, Calcium phosphates (CaPs), including hydroxyapatite (HAp) and tricalcium phosphate (TCP) or combinations thereof.

In one or more methods described herein the non-friable fibers comprise bio-neutral fibers, bio-neutral and medically functional fibers, bio-absorbable fibers, bioactive and bio-absorbable fibers, or combinations thereof.

In one or more methods described herein the non-friable fibers comprise polyolefin medical grade fiber, absorbent medical grade cotton or silk, polycaprolactones, polylactides, alginate, collagen, hyaluronic acid, silk fibroin, crosslinked chitosan, or combinations thereof.

In one or more methods described herein the first layer or at least one layer of non-friable fibers is a scrim for supporting the friable material and in one or more embodiments, the third layer is a second scrim for surrounding the friable material.

In one or more methods described herein a substantially solid material is encapsulated or otherwise supported by non-friable fibers and/or layers of non-friable fibers. The substantially solid material may be provided as a continuous material in part or in whole. The substantially solid material may also be provided in discrete pieces. In one or more of the methods described herein the substantially solid material comprises a fiber, a friable material, a friable fiber, a bioabsorable friable fiber, or a combination thereof.

In one or more of the articles or methods described herein discrete pieces of substantially solid material have overall dimensions are less than 1× an average length of the non-friable fibers in any at least one circumferential measurement.

In one or more of the articles or methods described herein discrete pieces of substantially solid material having at least one circumferential measurement that is at least 2× a shortest circumferential measure of at least 20% of the non-friable fibers.

Another aspect of the present disclosure relates to comprising a friable material supported by a non-friable material. The article may be used in tissue regeneration or wound care. The article has one or more layers of non-friable fibers surrounding non-friable material fibers wherein at least some of the non-friable fibers are oriented in a Z plane and at least some of the non-friable fibers are oriented in an XY plane such that the non-friable materials are surrounded by the non-friable fibers.

Yet another aspect of the present disclosure relates to an article comprising a friable material enclosed with a non-friable material. The article may be used in tissue regeneration or wound care. The article includes a bottom layer comprising a woven or non-woven layer of non-friable fibers wherein at least some of the fibers are oriented in an XY plane and a middle layer comprising friable material. A top layer comprising a woven or non-woven layer of non-friable fibers covers the second layer and wherein at least some of the fibers are oriented in the XY plane. The bottom layer and the top layer are secured together around the middle layer by at least some of the fibers of each of the bottom and top layer oriented in a Z plane to enmesh at least some fibers of the bottom layer and the top layer for securing the layers together.

In one more article described herein, the friable fibers comprise bioactive glass.

In one or more article described herein, the fibers orientated in the Z plane were needle punched to change orientation from the XY plane to the Z plane.

### BRIEF DESCRIPTION OF THE DRAWINGS

FIG. 1 illustrates a friable material layer being set on a bottom scrim.

FIG. 2 illustrates a top scrim being applied over the friable material and bottom scrim.

FIG. 3 is an image of enclosed friable material according to the methods described herein.

FIG. 4 is an image of a cross-section of the sample shown in FIG. 3.

FIG. 5 is an image of a cross-section of a portion of the sample shown in FIG. 3 where a top and bottom scrim have been secured together via methods described herein

FIG. 6 is a cross-section of the portion of FIG. 5 being separated to illustrate the entanglement of fibers in a z-direction.

FIG. 7 is a further illustration of fiber entanglement in the z-direction.

FIG. 8 illustrates a method of containing friable material.

FIG. 9 illustrates an arrangement of contained friable material.

FIG. 10 illustrates an arrangement of contained friable material.

FIG. 11 illustrates a tool for containing the friable material.

FIG. 12 illustrates a method of containing the friable material.

FIG. 13 illustrates a contained friable material.

FIG. 14 is a cross-sectional view of the contained friable material.

FIG. 15 illustrates a method of making a roll for cutting into bone implant shapes according to the present disclosure.

FIG. 16 illustrates an article produced according to the present disclosure.

FIG. 17 illustrates articles produced according to the present disclosure.

FIG. 18 illustrates articles produced according to the present disclosure.

FIG. 19 illustrates a method of needle punching a blend of material according to the present disclosure.

FIG. 20 illustrates a method of forming an article for use as a deep wound insert according the present disclosure.

FIG. 21 illustrates a method of forming an article for use as a deep wound insert according the present disclosure.

FIG. 22 illustrates an article formed according to the present disclosure.

### DETAILED DESCRIPTION

Methods described herein produce an article that is a cohesive material effectively containing a friable material. In one embodiment, the article comprises two or more layers of non-woven or woven fabric surrounding the friable material. In another embodiment, the article comprises two or more discrete layers of non-friable fibers surrounding the friable material. In yet another embodiment, the article comprises friable and non-friable fibers blended or distributed in a homogenous mixture.

Methods according to the present disclosure include encapsulating, containing, enmeshing and/or otherwise surrounding loose friable material within a network of fibers. The network of fibers may be felted to secure to two or more layers of fibers together. Moreover, a portion of a surface area of an assembly comprising one or more layers of non-friable fibers and a layer of friable fibers, or substantially across the surface area of the assembly may be felted to form an article. What is meant by the term “felted” as used throughout this disclosure is akin to felting or making into felt, or matting together such that the fibers described herein are caused to adhered and mat together to form a sort of cloth comprised of the fibers as described herein. To accomplish felting, a felting needle is used. Felting needles as defined herein may have a split pointed (forked) end for catching fibers to push the fibers through other fibers thereby entangling the fibers. The felting needle(s) may also have notches or barbs, positioned along the needle's shaft to catch fibers as the needle is pushed through but such barbs or notches configured so as not to catch the fibers as the needle is pulled back through the fibers. Of course felting needles may have both a split pointed end and barbs or notches.

As disclosed herein, friable material is a material that is useful for tissue growth or wound healing in vitro or in vivo and loose fibers of the friable material or chunks of the friable material is encapsulated, enmeshed, or surround by or within a network of fibers such as non-friable fibers and/or felted fibers. The disclosure herein refers to both friable fibers and a mass of friable fibers, crystals, particles and a solid piece of material provided in a selected shape such as a chunk of fibers having a pre-selected overall form for use.

Methods according to the present disclosure include layering a mass or quantity of the friable material with a fiber or a fibrous nonwoven material. In one embodiment, the method also includes pre-blending the friable material with binding fibers such as non-friable material fibers, and laying the blend down in a single layer.

The assemblies, whether comprising two or more layers of woven or nonwoven fibers and the friable material therebetween, two or more discrete layers of non-friable material surrounding the friable material or a layer of a blend of fibers of friable and non-friable material, the fibers in the layers are essentially arranged and orientated in an X-Y plane. A barbed felting needle is used to punch through the entire thickness of the assembly to catch and pull fibers from the X and Y plane in the Z direction to orient at least some of the fibers into the Z-plane, thus entangling the fibers and securing two or more layers of material together or otherwise forming a cohesive layer of non-woven fibers.

As used throughout this disclosure, the term “friable material” refers to material that easily falls apart, breaks into small bits, or separates making it difficult to handle or apply in a given application unless the friable material is contained.

Examples of friable materials suitable for the methods and articles described herein and used to promote wound healing and tissue growth include but not are limited to, bioactive glass, also referred to throughout this disclosure as “Bioglass,” which is a glass compositionally comprising SiO₂, CaO, Na₂O, and P₂O₅. The amounts of each component in the bioactive glass may vary as the Bioglass is tailored to use with different tissues. In one example, the bioactive glass or Bioglass comprises approximately 45 wt % SiO₂, 24.5 wt % CaO, 24.5 wt % Na₂O, and 6.0 wt % P₂O₅ and additional friable materials include but are not limited to unmodified chitosan, Calcium phosphates (CaPs), including hydroxyapatite (HAp) and tricalcium phosphate (TCP) or combinations thereof.

As used throughout this disclosure, the term “fiber” refers to material having a shape wherein one dimension is significantly larger than the other two dimensions and where the material is sufficiently flexible so as to enable entanglement with other fibers creating a three-dimensional network with sufficient strength to deliver the friable material for use in a given application.

Examples of non-friable material fibers suitable for the methods and articles described herein include, but are not limited to bio-neutral fibers such as polyolefin medical grade fiber, bio-neutral and medically functional fibers such as absorbent medical grade cotton or silk, bio-absorbable fibers such as or polycaprolactones and polylactides, and bioactive and bio-absorbable fibers, such as alginate, collagen, hyaluronic acid, silk fibroin, crosslinked chitosan, as well as combinations thereof and/or like fibers.

As used throughout this disclosure, the XY plane, X axis or Y axis, and Z plane or Z axis refer to the Cartesian coordinate where coordinates are often denoted by the letters X, Y, and Z and the axes may then be referred to as the X-axis, Y-axis, and Z-axis, respectively. Then the coordinate planes can be referred to as the XY-plane, YZ-plane, and XZ-plane where the xy-plane is horizontal and the z-axis points up (vertical plane).

The articles described herein are formed using a method referred to as felting. As used throughout this disclosure, the term “felting” refers to the process of producing a felted article. A mechanics operation is used to interlock fibers together to produce a non-woven article or “fabric” of sorts where the article is composed of interlocked fibers. While a method of needle punching for felting of the fibers to form a cohesive, non-woven article is described herein, other methods of felting or producing a mat of fibers may be used.

While needle felting or needle punching is a known fiber binding technology, the methods described herein with the materials described herein results in an improved, safe, cost-effective method of enclosing or surrounding friable material that eliminates the need for additional materials such as adhesives or solvents, which are not desirable components in materials for most medical applications. The methods described herein also do not require water, which can change or disrupt many friable materials used for wound healing or tissue growth promotion. The methods described herein also do not require sewing, which is a hand intensive, hygienically undesirable process for materials that will be introduced into the body. Further, the methods described herein can be used to densify and increase the strength of a fiber containing a blend of discrete materials.

The friable material may be provided as a substantially solid material that is encapsulated or otherwise supported by non-friable fibers and/or layers of non-friable fibers. The substantially solid material may be provided as a continuous material in part or in whole or the substantially solid material may additionally or alternatively be provided in discrete pieces. The substantially solid material comprises a fiber, a friable material, a friable fiber, a bioabsorable friable fiber, or a combination thereof.

For example, when discrete pieces of substantially solid material are provided, the pieces have overall dimensions are less than 1× an average length of the non-friable fibers in any at least one circumferential measurement.

The discrete pieces of substantially solid material may have at least one circumferential measurement that is at least 2× a shortest circumferential measure of at least 20% of the non-friable fibers.

Further, felting the fibers as described herein produces as article that can be provided in various selected lengths, widths, densities and thicknesses. The articles retain and wick water, oil, blood, and other liquids. The articles are sufficiently felted to resist wear and tearing and hold its edges such that the article will not unravel when cut.

The articles described herein and/or produced by the methods described herein may be provided as a continuous non-woven web that can be handled without disintegrating and wherein the web can be cut and/or shaped into forms useful for medical applications. The articles are felted in that the articles are non-woven materials of interlocked fibers.

It is also contemplated that the articles described herein may comprise the layers of fiber placed to surround a pre-formed shape or pattern of friable material (e.g., the friable material may be provided as a ball or shaped into a cylinder or rod or other shape having one or more dimensions suitable to implant in a wound or to guide cell growth in the shape of an organ or bone.) and wherein the fiber is secured and entangled around the friable material as the fibers are needle felted through at least two layers of fiber in locations surrounding the friable material. A portion of the surface area of the layered assembly of the article or substantially the surface area of the layered assembly may be felted to form the article. Felting may be done by needle punching or other methods of enmeshing fibers. For example, a process using felting needs in a 3-dimensional manner may be utilized.

The area subject to felting, or needle punching, may include outside the periphery of the friable material, such as a perimeter area with respect to the friable material between the layers, thereby securing the friable material between the two layers of non-woven mat. Additionally, or alternatively, substantially the entire surface area of the assembly may be needle punched including the assembly having the friable material therein. This includes both the assembly where the friable material is present in only a portion of the assembly and when the friable material is present across substantially the entire assembly. For purposes of containment, non-fibrous scrim or film of any material that can be punched through may also be layered between the outermost fibrous layers and will be held in place by the fibers drawn and entangled in the z-direction.

The articles described herein may be used in a variety of medical applications including, but not limited to, wound bandages or packing for primary wound treatment in the field; permanent wound treatments, packing, or scaffolding that remains in place and is eventually reabsorbed by the body; permanent tissue growth scaffolding that is designed to regrow complex tissue and provide support to the surrounding tissue during regrowth.

The methods described herein may be carried out to provide a range of articles having various properties including but not limited to thickness, pliability, density, size, ratio of friable material to fiber; degree of felting or combinations thereof. When articles are minimally felted in the z direction the resulting article is loftier or a softer article. When the article is densely felted in the z direction the article is a structurally supportive article with increased tensile properties.

The extent of felting, or needle punching, carried out may be pre-selected based on the properties of the article sufficient for its end use as noted directly above. In some embodiments, the article should be felted sufficiently to produce an article having interlocked fibers for strength.

### Illustrative Example 1

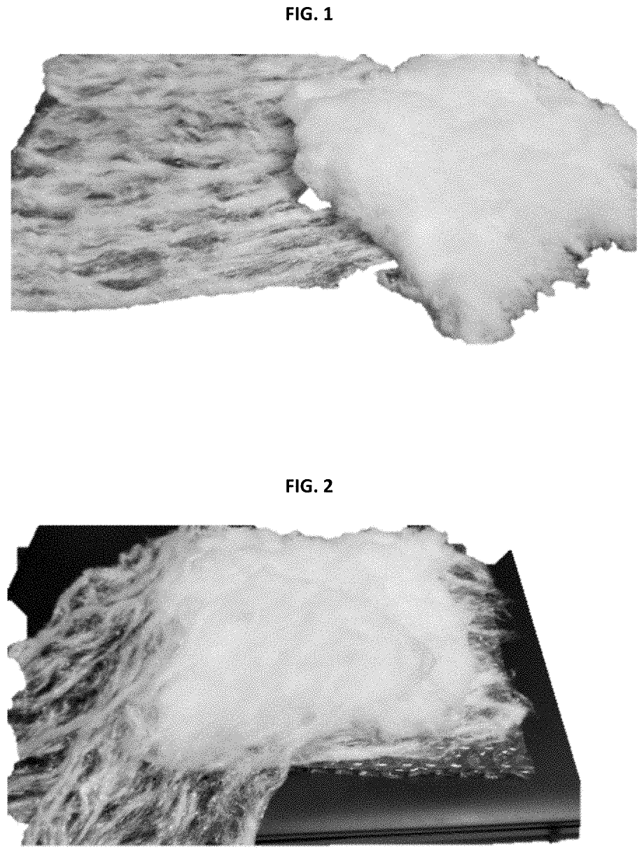

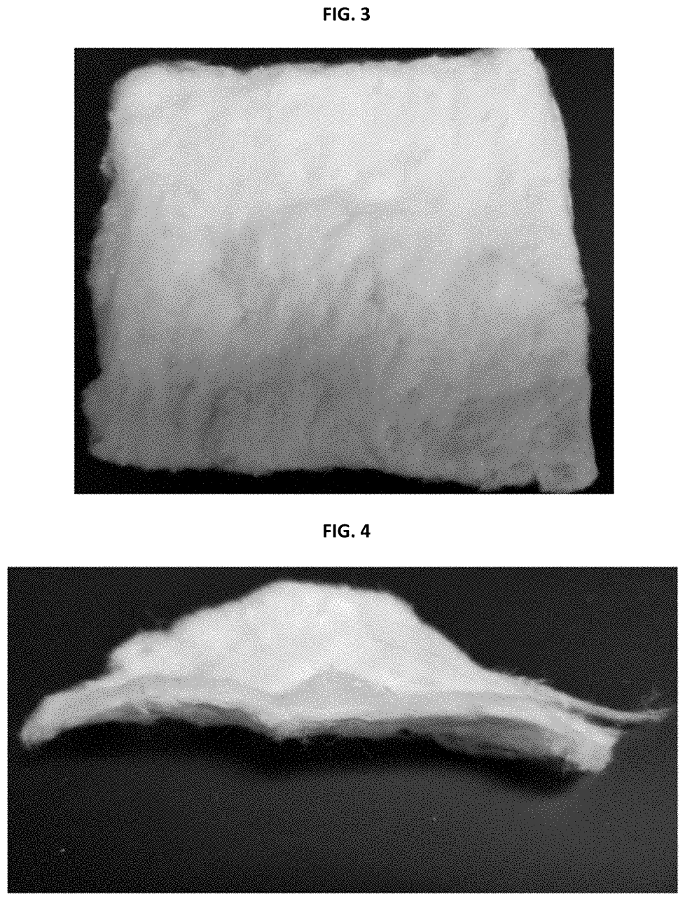

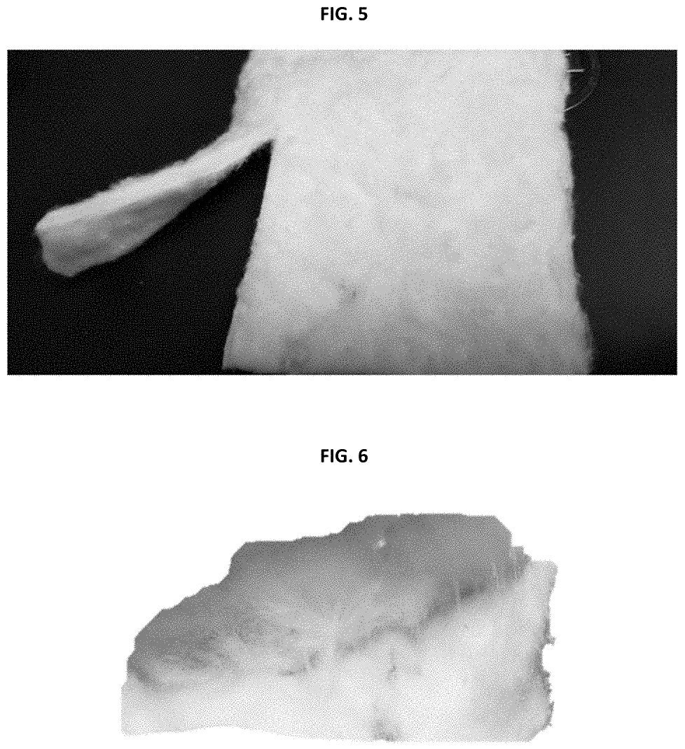

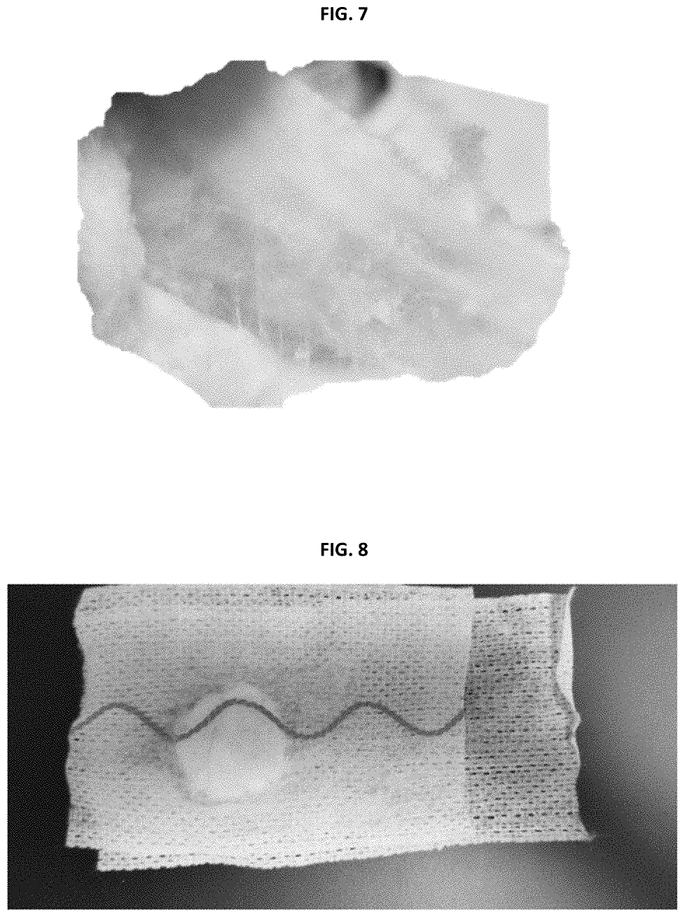

Referring to FIGS. 1-7, a one-inch thick layer of bio-glass formed by a blowing process is placed between a top and a bottom scrim, the scrims comprising cotton fiber having fibers oriented substantially in the XY plane. The scrims each have a thickness less than the layer of bio-glass. The assembly, including the bottom scrim fibers, the bio-glass, and the top scrim fibers is then needle felted. At least some of the fibers of the top and bottom scrim are pulled into the Z plane along the Z axis such that the fibers extend into or out of the XY plane to secure the top and bottom scrim layers together at a selected level of felting and thus encapsulating or surrounding the bio-glass between the top and bottom scrim. As illustrated in FIGS. 1-2, the arrangement was placed on a substrate having numerous small air cushions such as bubble wrap, which allowed the needle(s) to completely pierce through the assembly during needle felting. The encapsulated and felted sample is shown in FIG. 3 where the sample also illustrates different levels of felting including denser felting near the bottom of FIG. 3. As illustrated in FIGS. 4 and 5, the sample can be folded over in half and subject to further needle punching which provides yet further densification producing a denser article. FIGS. 6-7 illustrate the enmeshing of the fibers of the top and bottom scrim where the fibers are moved into orientation in the Z plane.

### Illustrative Example 2

As illustrated in FIG. 8, a portion of bio-glass is compiled in a rounded shape and placed on, and approximately in the center of a first scrim comprising cotton gauze and a second scrim of cotton gauze is placed over the bio-glass and first scrim to hold the bio-glass therebetween. A felting needle is used to punch directly through a perimeter of the first and second scrim layers where the perimeter is defined as the edge or area around the bio-glass in order to encapsulate the bio-glass between the first and second scrim. Needle punching securely fastens the first and second scrim together for holding the bio-glass in place.

### Additional Examples

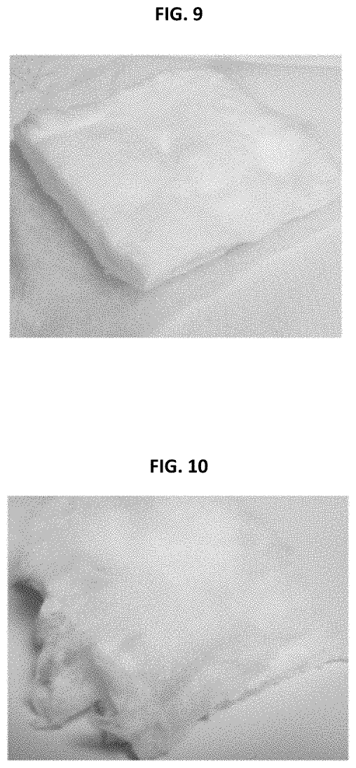

Referring to FIG. 9, bio-glass may be encapsulated in one or more layers of alginate fiber with needle punching. In FIG. 10, multiple layers of bio-glass may be encapsulated in alginate fiber.

In some embodiments wherein calcium alginate fibers are used for the fiber or scrim layer(s), the calcium alginate fibers can be produced by shredding a commercially available alginate pad to provide fibers for layering between and around the friable material such as bio-glass. The fibers are then needle punched thereby attaching to or enmeshing the fibers with each other and surrounding the bio-glass. The needle punching of the shredded calcium alginate produces a non-woven textile type of pouch to hold the bio-glass.

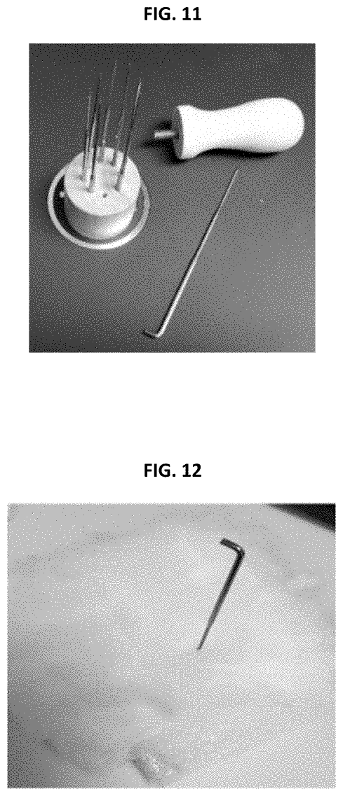

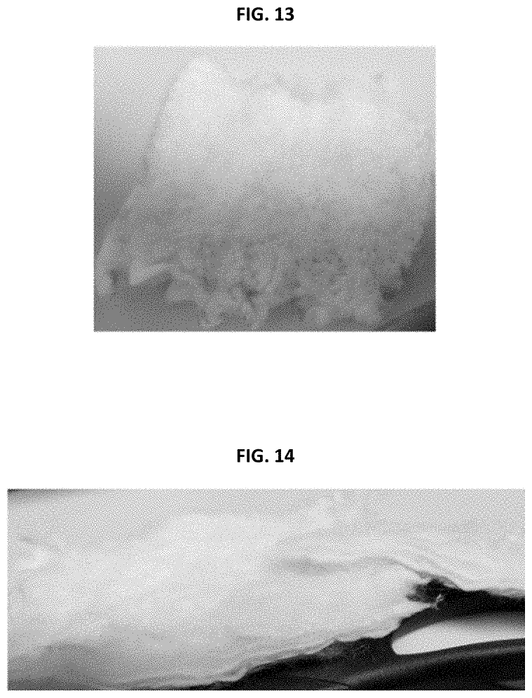

FIG. 11 illustrates one device for needle punching or needle felting. A needle having one or more barbs on its length is pushed completely through one or more layers of fibers or in some embodiments one or more layers of fibers and friable material to catch and pull fibers from a first orientation such as in the XY-plane to a second orientation such as in the Z-plane. The layers are punched repeatedly, all the way through, to draw fibers into a different orientation and to create a three-dimensional entangled fiber mesh which holds the friable material in place. The article is thus a non-woven article.

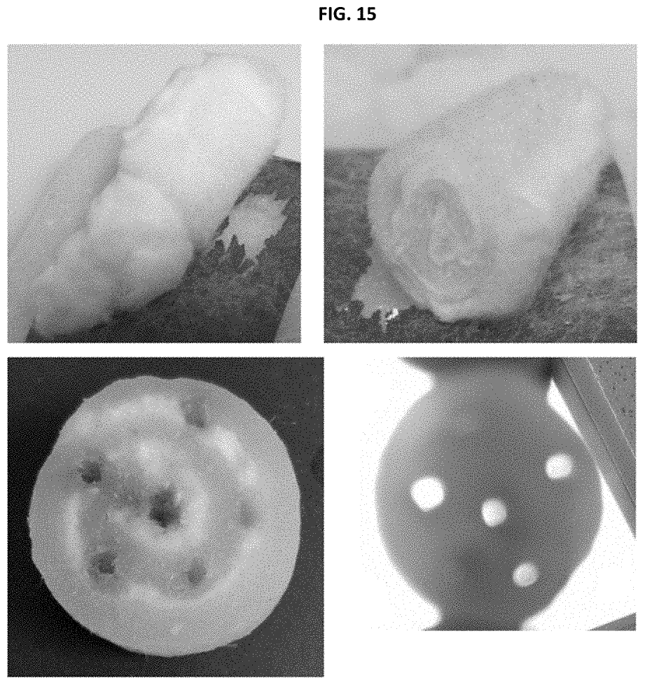

As illustrated in FIG. 15, articles described herein and/or produced by the methods described herein may be formed into a roller and cut into selected shapes including bone implant shapes. For example, a layer of Bioglass may be rolled up with a layer of alginate non-woven web. The roll is then needle punched to fasten the layers together, create a denser and stronger form, and to retain a cylindrical shape of a bone implant. Discs are then cut off the roll with a knife and a laser can be used to punch holes typical of a bone implant. The implant disk may then be soaked in water to demonstrate what happens when the implant comes into contact with body fluids. FIG. 15 illustrates how the alginate swells up filling the empty areas with gel. This implant model is made of entirely bioabsorbable tissue growth promoting materials.

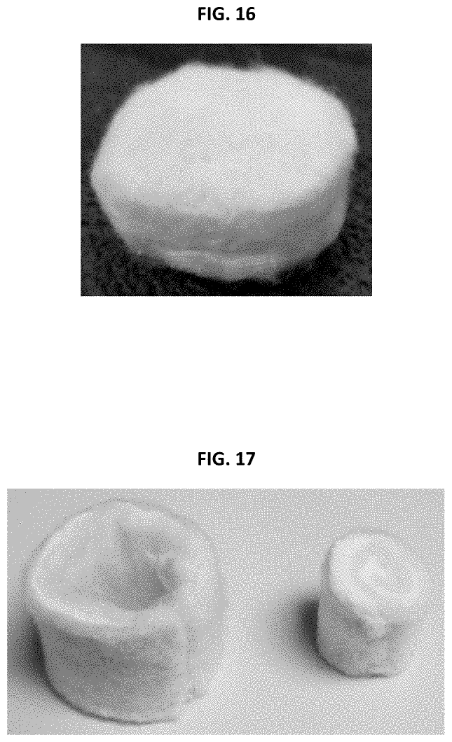

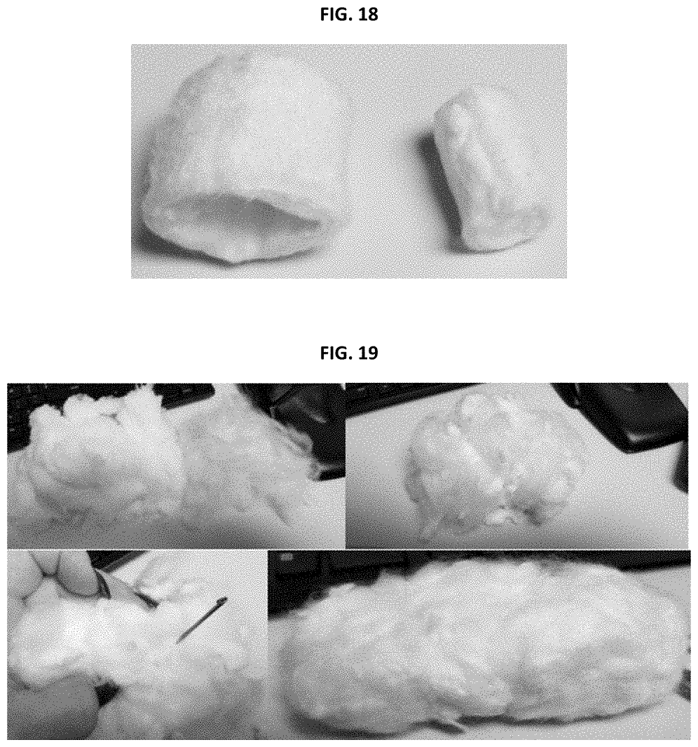

Referring now to FIGS. 16-18, various possible alginate bioglass implants can be made by the methods described herein. In FIG. 16, an article is produced by needle punching alternating layers of alginate and bioglass cut into a disk. In FIGS. 17-18, needle punched shapes made of layers of bioglass and alginate are illustrated.

In FIG. 19 needle punching a blend of alginate and bioglass is illustrated.

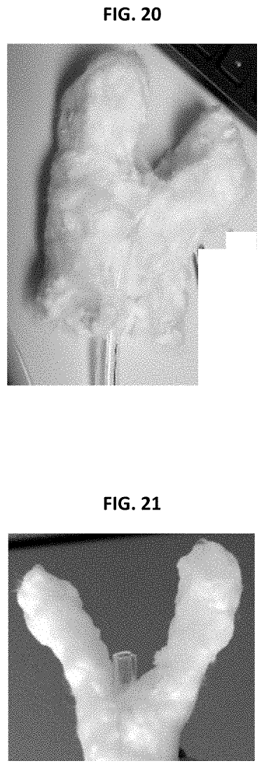

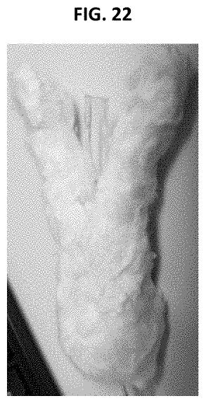

FIGS. 20-22 illustrate fibers that were randomly blended, then needle punched sufficiently to hold them together. The fibers can then be cut into a shape of a deep wound insert containing a fluid tube used to draw off excess body fluid and allow therapeutic solutions to be inserted. The openings around the tube can be needle punched shut and the whole insert then needle punched further to densify and strengthen it. FIG. 22 illustrated the article swelled with water around the tube to demonstrate how it would behave immersed in body fluid. This whole deep wound insert, except for the tube, is constructed of bioabsorbable tissue growth promoting materials.

In some embodiments a woven or non-woven layer is placed on both sides of the friable material and the assembly is then needle punched to secure the friable material between the layers which produces a non-woven mat. The woven or non-woven layers may be referred to as scrims and the scrims have dimensions greater than the quantity or shape of the friable material placed therebetween such that the friable material is within only a portion of the assembly. Thus the non-woven mat may have the friable material across only a portion of the non-woven mat surface area. In another embodiment, the friable material may be provided in a shape or quantity about the same dimensions as the woven or non-woven layers or may be spread across substantially the surface area of the woven or non-woven layers such that the friable material is present across substantially the entire non-woven mat when produced.

It is also contemplated and within this disclosure that one or more layers of woven or non-woven material is felted with the friable material such that the article produced is a cohesive non-woven mat with the friable material felted therein, as opposed to the friable material being encapsulated between two discrete layers of woven or non-woven fibers.

Depending on the subsequent or end use of the friable material, the non-woven mat may be made of a material suitable for and related to the subsequent or end use.

Although the present disclosure has been described with reference to preferred embodiments, workers skilled in the art will recognize that changes may be made in form and detail without departing from the spirit and scope of the disclosure.

1. A method for producing an article with a friable material enclosed therein, the method comprising:
providing a first layer comprising non-friable fibers;
providing a second layer comprising friable material on top of the first layer;
providing a third layer comprising non-friable fibers on top of the first and second layer; and
pushing a felting needle repeatedly through at least the first and third layer to secure the first and third layer together with the second layer therebetween such that the first and third layer are secured around the second layer.

2. The method of claim 1 wherein the first and third layer each have a width and a length greater than a width and a length of the second layer.

3. The method of claim 1 wherein pushing the felting needle comprises needle felting the first and third layer together by pushing fibers in the first and second layer into orientation along a Z-axis to enmesh the fibers of the respective first and third layers for securing the first and third layers together.

4. The method of claim 1 wherein a density of the article is controlled by selecting the number of times the felting needle is pushed through one or more selected areas of the article entangling the non-friable fibers to pull the bulk of the material together and wherein the density may be the same of different across the article.

5. The method of claim 1 wherein the biocompatible fibers of the first and third layer are the same or different biocompatible fibers.

6. The method of claim 1, wherein the friable material comprises bioactive glass, unmodified chitosan, Calcium phosphates (CaPs), including hydroxyapatite (HAp) and tricalcium phosphate (TCP) or combinations thereof.

7. The method of claim 1, wherein the non-friable fibers comprise bio-neutral fibers, bio-neutral and medically functional fibers, bio-absorbable fibers, bioactive and bio-absorbable fibers, or combinations thereof.

8. The method of claim 1, wherein the non-friable fibers comprise polyolefin medical grade fiber, absorbent medical grade cotton or silk, polycaprolactones, polylactides, alginate, collagen, hyaluronic acid, silk fibroin, crosslinked chitosan, or combinations thereof.

9. The method of claim 1, wherein the first layer is a first scrim for supporting the friable material and the third layer is a second scrim for surrounding the friable material.

10. A method of producing a non-woven cohesive fiber article comprising:
providing one or more layers of non-friable fibers and a layer of friable materials wherein at least the non-friable fibers are substantially oriented approximately in the XY plane defined by the substantial original orientation of a majority of the fibers;
pushing a felting needle through a thickness defined by the layer or layers of the non-friable fibers and the layer of the friable fibers to push at least a portion of the non-friable fibers to change the orientation of the portion of the non-friable fibers to orientation approximately in the Z plane relative to an original orientation of the majority of the fibers for securing the friable fibers within a 3 dimensional mesh of non-friable fibers.

11. The method of claim 10, wherein the friable fibers comprise bioactive glass, unmodified chitosan, Calcium phosphates (CaPs) such as hydroxyapatite (HAp) or tricalcium phosphate (TCP) or combinations thereof.

12. The method of claim 10, wherein the non-friable fibers comprise polyolefin medical grade fiber, absorbent medical grade cotton or silk, polycaprolactones, polylactides, alginate, collagen, hyaluronic acid, silk fibroin, crosslinked chitosan, or combinations thereof.

13. An article with a friable material supported by a non-friable material, the article comprising:
one or more layers of non-friable fibers surrounding friable material fibers wherein at least some of the non-friable fibers are oriented in a Z plane and at least some of the non-friable fibers are oriented in an XY plane such that the friable material fibers are surrounded by the non-friable fibers.

14. The article of claim 13 wherein the article is configured for use in bandages for wounds, packing for primary wound treatment in the field, permanent wound treatments, packing, in vivo scaffolding or combinations thereof.

15. The article of claim 13, wherein the friable fibers comprise bioactive glass and wherein the fibers orientated in the Z plane are felted to change orientation from the XY plane to the Z plane.

16. The article of claim 13 wherein the one or more layers comprise:
a bottom layer comprising a woven or non-woven layer of non-friable fibers wherein at least some of the fibers are oriented in an XY plane;
a middle layer comprising friable material;
a top layer comprising a woven or non-woven layer of non-friable fibers wherein at least some of the fibers are oriented in the XY plane; and
wherein the bottom layer and the top layer are secured together around the middle layer by at least some of the fibers of each of the bottom and top layer oriented in a Z plane to enmesh at least some fibers of the bottom layer and the top layer for securing the layers together.

17. The article of claim 16, wherein the friable fibers comprise bioactive glass, unmodified chitosan, hydroxyapatite (HAp), tricalcium phosphate (TCP), or combinations thereof.

18. The article of claim 16, wherein the non-friable fibers comprise medical grade polyolefin fibers, absorbent medical grade cotton or silk, polycaprolactones, polylactides, alginate, collagen, hyaluronic acid, silk fibroin, crosslinked chitosan, or combinations thereof.

19. The article of claim 13 wherein the friable material fibers are provided in a plurality of discrete pieces each having overall dimensions that are less than 1× an average length of the non-friable fibers in any at least one circumferential measurement or have at least one circumferential measurement that is at least 2× a shortest circumferential measure of at least 20% of the non-friable fibers.

20. The article of claim 19 wherein the friable material fibers are secured by the non-friable fibers at one or more selected areas throughout the article and wherein the selected areas comprise edges of the article, center portions of the article or a combination thereof.
